(19) United States
(12) Patent Application Publication
(10) Pub. No.: US 2026/0000839 A1
(43) Pub. Date: Jan. 1, 2026

(54) VOICE INTEGRATED, MULTI VARIABLE, PLATFORM ADAPTABLE MEDICAL DOSE ASSIST MODULE WITH STRUCTURAL SAFETY FEATURES AND ENHANCED GUIDANCE

(71) Applicant: Srinivasan Ganti, Alameda, CA (US)

(72) Inventor: Srinivasan Ganti, Alameda, CA (US)

(21) Appl. No.: 19/320,802

(22) Filed: Sep. 5, 2025

(51) Int. Cl. A61M 5/315; A61M 5/31

(52) U.S. Cl. CPC A61M 5/31546; A61M 5/31511; A61M 5/31541; A61M 5/3158; A61M 2005/3126; A61M 2205/121; A61M 2205/18; A61M 2205/3592; A61M 2205/505; A61M 2205/581; A61M 2205/583; A61M 2205/585; A61M 2205/586; A61M 2205/8262; A61M 2230/005; A61M 2230/04

(57) ABSTRACT

A portable dose-assist module couples to a medication cartridge and provides on-device, voice-integrated guidance through a display and user controls (four-way toggle, rocker, microphone/button). A dosing engine fuses physiologic, behavioral, and contextual inputs (including optional sensor data and user-entered wellness, meals, activity, and concurrent medications) to generate therapy recommendations for user approval. Medication delivery is user-performed; the module does not automatically actuate a pump, solenoid, or plunger. The module operates independently without a smartphone interface by caching data on-device and synchronizing via Wi-Fi or through a cradle with a simplified keypad that also supports limited fallback input/output. A separation safety seal is located between the plunger assembly and the cartridge interface, outside the medication fluid path and remaining unpierced during cartridge changeover to reduce cross-contamination. The cartridge interface accepts a 3 mL, 100-unit O-ring-type cartridge. The platform supports multiple therapeutic categories.

### CROSS-REFERENCE TO RELATED APPLICATIONS

Not applicable.

### FIELD OF THE INVENTION

The disclosure relates to portable, cartridge-compatible drug-delivery assist devices and associated workflows for subcutaneous medications. More particularly, it concerns a platform-configurable dose-assist module with an on-device display and user-input controls (e.g., four-way toggle, rocker, and microphone/button); voice-integrated guided-intake and logging of lifestyle factors; and structural safety features including a separation seal. The module logs dose events via a plunger-linked sensor arrangement and provides therapy recommendations without initiating closed-loop delivery. The architecture supports multiple therapeutic categories—including, without limitation, endocrine, neurologic, cardiovascular, gastrointestinal, pain-management, and weight-management regimens—while operating independently of a smartphone by using on-device caching, Wi-Fi synchronization, and an optional cradle interface with a simplified keypad.

### BACKGROUND

Conventional connected drug-delivery solutions are commonly optimized for a single therapy and distribute core functionality across phone-dependent accessories or multi-component systems. These approaches can create reliability and accessibility challenges, including continuous smartphone dependence, limited on-device interaction, and fragmented records across different therapies. Existing pen-style systems may also lack structural barriers that reduce cross-contamination around the plunger and cartridge interface, and many interfaces do not adequately accommodate voice-integrated, multilingual guidance for users with visual, dexterity, or language constraints. Clinicians further face fragmented reporting that does not unify dosing, intake context, lifestyle information, and biometric inputs across multiple regimens.

### SUMMARY

In one aspect, a portable, platform-configurable medical dose-assist module includes: a housing including a cartridge interface configured to accept a medication cartridge; a control module containing a configurable display and at least one user-input device selected from the group consisting of a four-way toggle, a rocker switch, and a microphone/button, and combinations thereof; and a data/power port; a plunger assembly mechanically and electronically linked for dose logging; a separation safety seal located between the plunger assembly and the cartridge interface, positioned outside the medication fluid path and remaining unpierced during cartridge changeover, to reduce cross-contamination; and one or more processors executing a dosing engine that fuses physiologic, behavioral, and contextual inputs to generate therapy recommendations for user review and approval. The module does not automatically actuate a pump motor, solenoid, or a plunger to deliver medication; medication delivery, including any plunger actuation, is performed by the user.

The module operates independently without a smartphone interface by caching data on-device and synchronizing via Wi-Fi or through a cradle interface having a simplified keypad that enables manual entry of credentials and therapy-related values and provides limited fallback input/output when the module is nonresponsive. Guided-intake workflows prompt for wellness status, meal intent, activity, concurrent medications, and therapy-specific factors; voice-integrated lifestyle capture supports daily routines and adherence; and a nutrient-lookup workflow queries a local cache with selective cloud expansion when available. Clinician-facing reports can summarize guided intake and dosing history in exportable formats. The cartridge interface is configured to accept a 3 mL, 100-unit O-ring-type cartridge suitable for subcutaneous delivery.

The module is structurally distinct from replacement pen-cap accessories; the housing is not a replacement pen cap. The platform is therapy-agnostic and extends beyond diabetes to support multiple therapeutic categories.

In another aspect, a method for providing multi-condition medication dose assistance comprises receiving physiologic and user-supplied inputs, integrating the inputs with stored dosing instructions and therapy-specific parameters to generate a composite guidance output, and outputting a therapy recommendation for user approval without initiating closed-loop delivery.

In a further aspect, a non-transitory computer-readable medium stores instructions that, when executed by one or more processors, cause performance of the method set forth above.

### BRIEF DESCRIPTION OF THE DRAWINGS

FIG. 1—Exterior perspective view showing the barrel (100), display window (112), four-way toggle (114), data/power port (e.g., USB-C) (116), microphone/button (118), rocker switch (130), plunger paddle (150), and needle (170).

FIG. 2—Assembled exterior view illustrating the plunger paddle (150), display window (112), four-way toggle (114), rocker switch (130), microphone/button (118), data/power port (116), barrel (100), and needle (170).

FIG. 3—Exploded side view illustrating the plunger paddle (150), plunger assembly (140), barrel (100), threaded collar (120), separation safety seal (160), and needle (170).

FIG. 4—Side sectional view illustrating the display window (112), dosing engine (200), power source (210), separation safety seal (160), barrel (100), and plunger assembly (140).

FIG. 5—Front, side, and three-quarter views of the plunger paddle (150).

FIG. 6—User-interface flow diagram illustrating the power source (210), wellness inquiry (220), meal input (222), activity input (230), medication input (240), and therapy guidance output (272).

FIG. 7—Functional overview diagram illustrating the power source (210), wellness input (220), meal input (222), activity input (230), medication input (240), CGM/sensor interface (250), wearable or phone metrics (260), dosing engine (200), and therapy guidance output (272).

FIG. 8—Voice-driven nutrient lookup flow illustrating the microphone/button (118), local nutrient cache (280), optional cloud expansion module (290), portion prompt engine (270), and therapy guidance output (272).

FIG. 9—Security and permissions schematic illustrating the security module (300), remote wipe control (310), and alarm signaling path (372).

FIG. 10—Schematic of wearable- or phone-based inputs showing steps (350), heart rate (352), ECG/EKG (354), and blood pressure (356).

FIG. 11—Sectional view of cradle (320) illustrating pogo pin contacts (360), contact plates (362), raised side rails (364), keypad (366), and power/charging interface (368).

FIG. 12—Synchronization pathways illustrating the cradle interface (320) with keypad (366), Wi-Fi sync module (330), and Bluetooth sync module (340).

FIG. 13—Detail view of alarm switch (370).

FIG. 14—Exterior view illustrating raised side rails (364) and keypad (366).

FIG. 15—Exploded sectional view of the cradle illustrating pogo pin contacts (360) and corresponding contact plates (362), together with a power/charging interface (368).

FIG. 16—Side views of cradle showing flat base and angled base variant (358).

FIG. 17—Schematic of the separation alert pathway illustrating the CGM/sensor interface (250), cradle interface (320), and alert output (372).

FIG. 18—Schematic indicating cartridge compatibility; numerals omitted for clarity.

### REFERENCE NUMERAL SCHEDULE

100—Barrel
112—Display window
114—Four-way toggle
116—Data/power port (e.g., USB-C)
118—Microphone/button
120—Threaded collar
130—Rocker switch
140—Plunger assembly
150—Plunger paddle
160—Separation safety seal
170—Needle
200—Dosing engine
210—Power source
220—Wellness inquiry
222—Meal input
230—Activity input
240—Medication input
250—CGM/sensor interface
260—Wearable/phone integration interface
270—Portion prompt engine
272—Therapy guidance output
280—Local nutrient cache
290—Cloud expansion module
300—Security module
310—Remote wipe control
320—Cradle interface
330—Wi-Fi sync module
340—Bluetooth sync module
350—Steps sensor input
352—Heart rate input
354—ECG/EKG input
356—Blood pressure input
358—Angled base variant
360—Pogo pins
362—Contact plates
364—Raised side rails/barriers
366—Integrated keypad
368—Power input/charging interface
370—Alarm on/off switch
372—Separation alarm alert signal

### DETAILED DESCRIPTION OF THE INVENTION

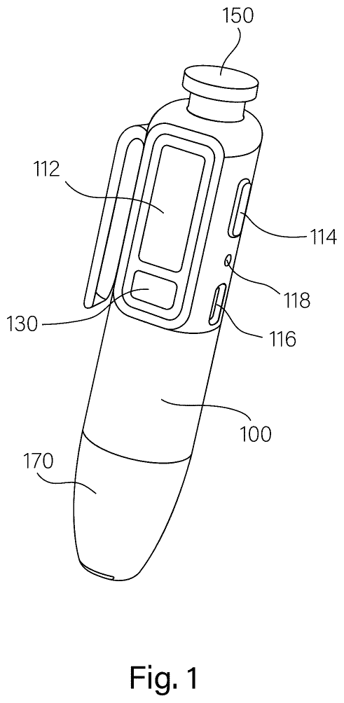

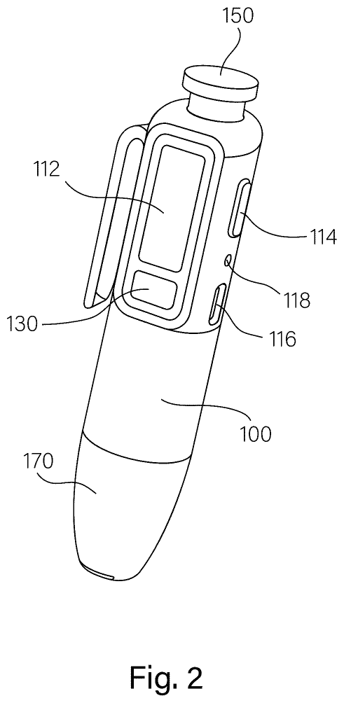

FIGS. 1-2—Exterior Views. The exterior views illustrate the module's primary user-facing components: display window (112), four-way toggle (114), data/power port (116), microphone/button (118), rocker switch (130), barrel (100), plunger paddle (150), and needle (170). These elements enable guided-intake interaction and manual dose administration while maintaining a compact, hand-held form factor. The display window (112) may be configured to render either a single high-contrast line for large-font readability or two lines for expanded information display.

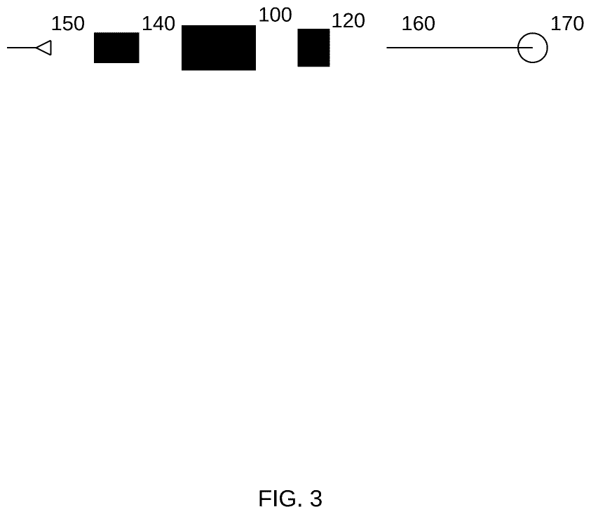

FIG. 3—Exploded View. From front to rear, the assembly includes the needle (170), threaded collar (120), cartridge body received within the barrel (100), separation safety seal (160), plunger assembly (140), and plunger paddle (150). The safety seal (160) is formed of a medical-grade elastomer and is configured to reduce ingress or egress of biological material into the plunger cavity, thereby mitigating cross-contamination risk during cartridge use and handling.

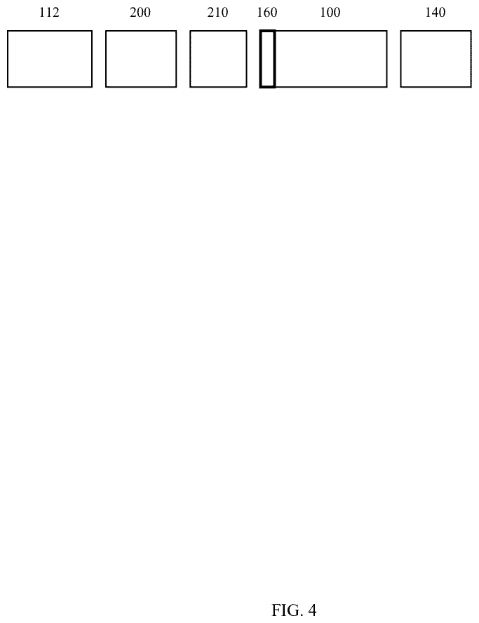

FIG. 4—Side Sectional View. The side sectional view shows the relationship between the display window (112), dosing engine (200), power source (210), separation safety seal (160), barrel (100), and plunger assembly (140). In various embodiments, the electronics are arranged to maintain serviceability while preserving the sterile boundary provided by the seal (160).

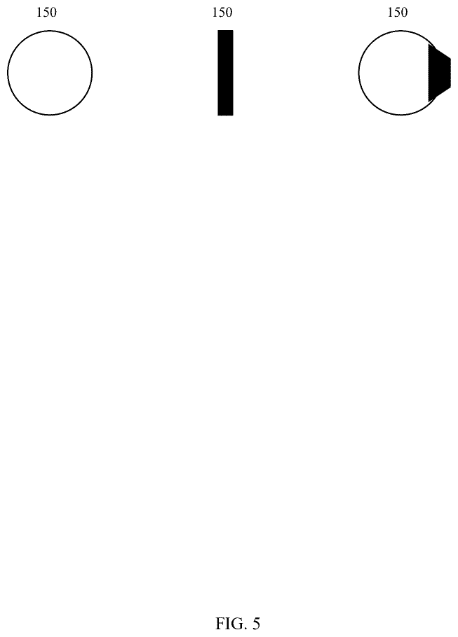

FIG. 5—Plunger Paddle. Front, side, and three-quarter perspectives of the plunger paddle (150) are shown. The paddle may incorporate ergonomic shaping and tactile features to facilitate one-hand actuation and to assist users with tremor or reduced dexterity.

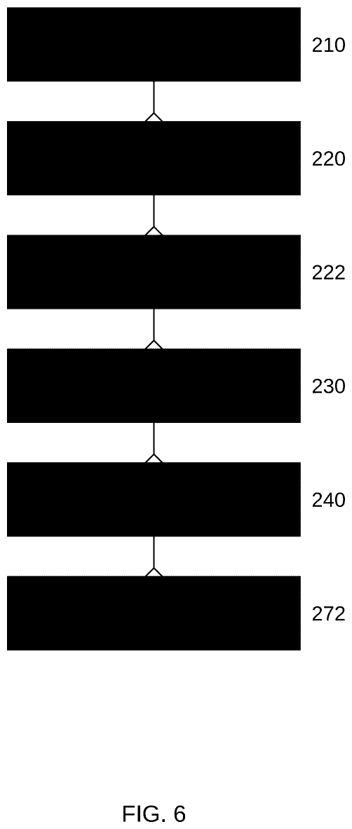

FIG. 6—User-Interface Flow. This figure depicts a guided-intake flow in which the power source (210) enables prompts across wellness inquiry (220), meal input (222), activity input (230), and medication input (240), culminating in a therapy guidance output (272).

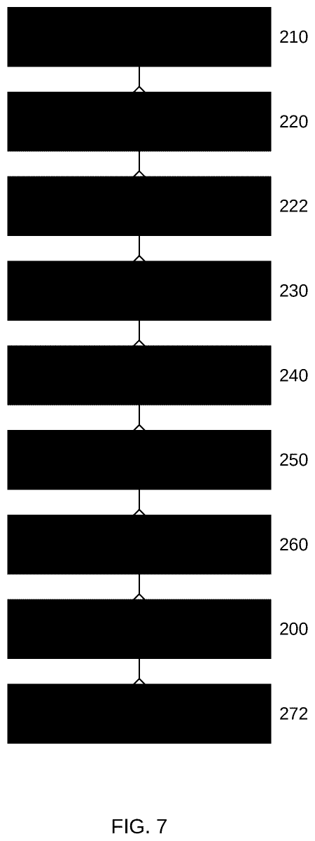

FIG. 7—Functional Overview. Sensor and context inputs, including wearable or phone metrics (260) and an optional CGM/sensor interface (250), are integrated by the dosing engine (200) to produce a therapy guidance output (272).

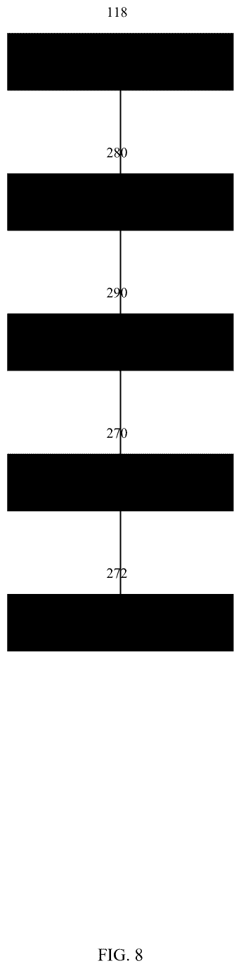

FIG. 8—Nutrient Lookup. Voice-driven lookup uses the microphone/button (118) to query a local nutrient cache (280) and, when available, a cloud expansion module (290). A portion prompt engine (270) assists with portion selection, and results contribute to the therapy guidance output (272).

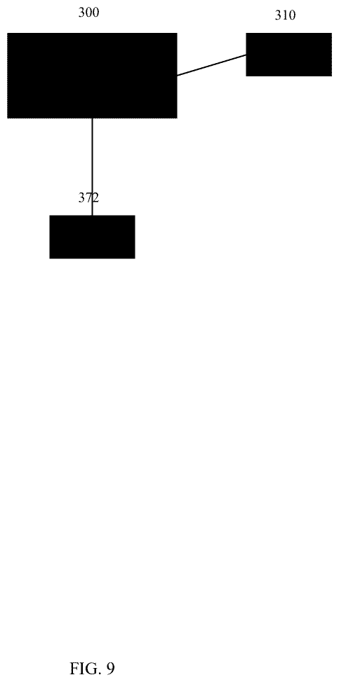

FIG. 9—Security and Permissions. A security module (300) manages access control and firmware integrity. Remote wipe control (310) enables device-only or device-plus-cradle data wipes. An alarm signaling path (372) supports location-assist and separation alerts.

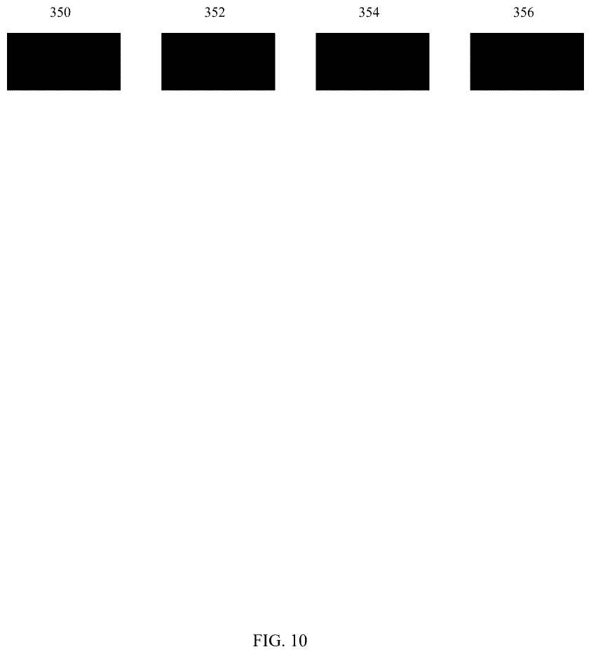

FIG. 10—Wearable/Phone Inputs. Example wellness inputs include steps (350), heart rate (352), ECG/EKG (354), and blood pressure (356). These signals may be optionally incorporated as contextual inputs without overriding stored dosing instructions.

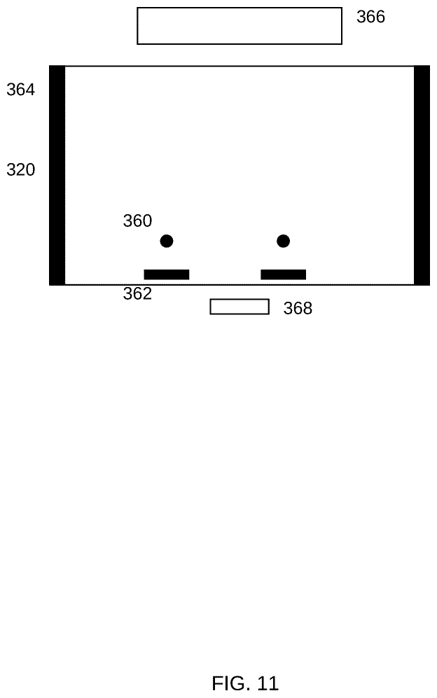

FIG. 11—Cradle Sectional. The cradle interface (320) includes pogo pins (360) that mate with contact plates (362), raised side rails (364) for alignment and retention, a keypad (366), and a power/charging interface (368).

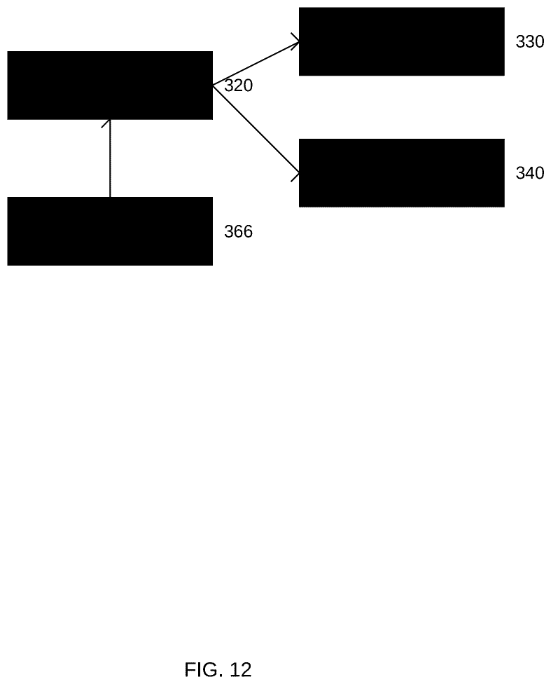

FIG. 12—Synchronization Pathways. The module can synchronize via the cradle interface (320) and keypad (366), Wi-Fi sync module (330), or Bluetooth sync module (340). The module operates independently without a smartphone interface by caching data on-device and performing deferred synchronization when a connection becomes available. The cradle provides a simplified keypad configured to enable manual entry of time adjustments, biometric or therapy-related values, Wi-Fi credentials, and healthcare-provider link codes, and can assume limited input/output functionality including error prompts and data recovery when the module is nonresponsive.

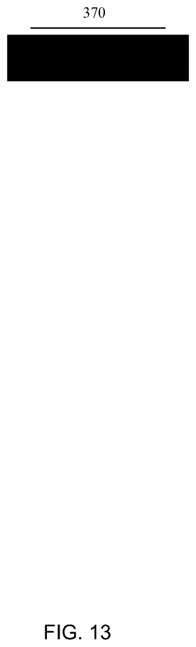

FIG. 13—Alarm Switch Detail. The alarm switch (370) provides ACTIVE and SILENCE detents. Safety-critical alerts can be configured to bypass the SILENCE state.

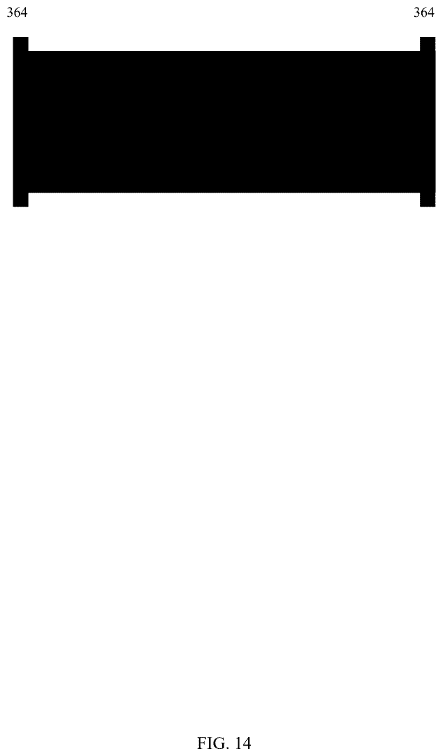

FIG. 14—Exterior Cradle Features. Raised side rails (364) provide lateral guidance and retention of the module within the cradle, and the keypad (366) enables manual entry for setup and diagnostics.

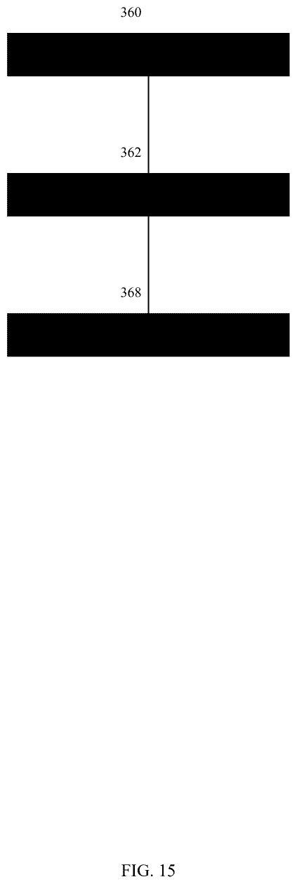

FIG. 15—Exploded Cradle Section. The cradle illustrates pogo pin contacts (360) and corresponding contact plates (362), together with a power/charging interface (368) for data and power coupling.

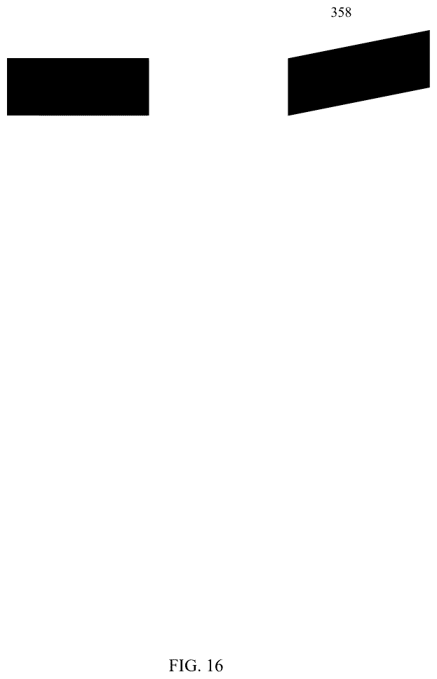

FIG. 16—Base Variants. Side views of a flat base and an angled base variant (358) are shown to illustrate alternative docking geometries.

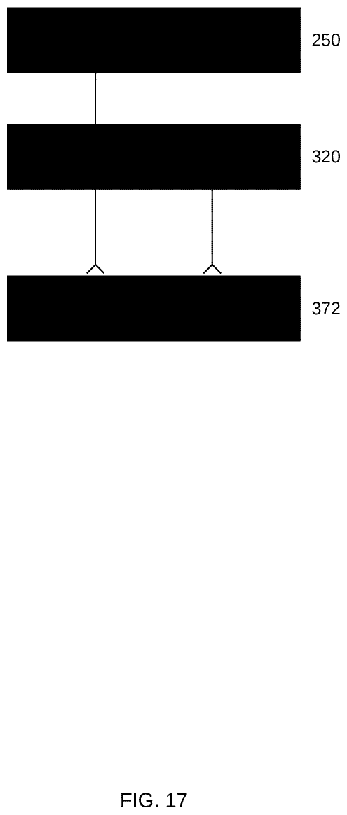

FIG. 17—Separation Alert Pathway. The schematic illustrates signaling among a connected sensor interface (250), the cradle interface (320), and an alert output path (372).

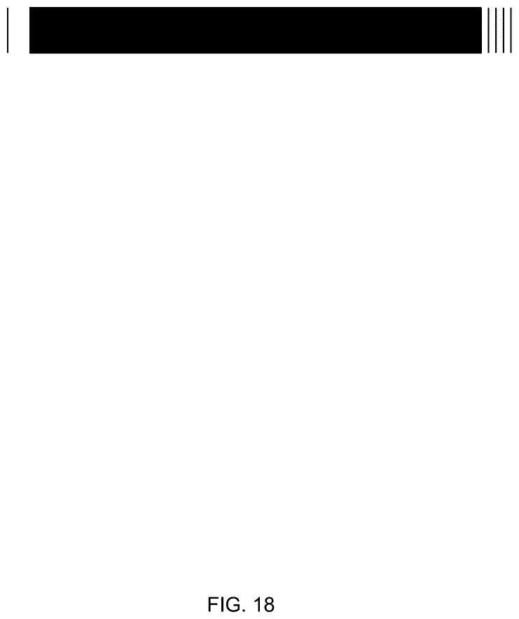

FIG. 18—Cartridge Compatibility. FIG. 18 schematically indicates compatibility with medication cartridges. In various embodiments, the cartridge interface is configured to accept a 3 mL, 100-unit O-ring-type cartridge suitable for subcutaneous delivery; specific cartridge geometry is not limiting unless otherwise specified.

OPERATION. A medication cartridge is coupled to the barrel (100) and primed according to therapy-specific instructions. A disposable needle (170) is installed at the cartridge end. The user initiates guided intake via the microphone/button (118), four-way toggle (114), or rocker (130). The dosing engine (200) integrates stored dosing instructions with context inputs to generate a recommendation for user review and approval.

DOSE ADMINISTRATION. Medication delivery is performed by the user. The user confirms a suggested dose by pressing the plunger paddle (150), which advances the cartridge piston to deliver medication. Plunger-linked sensing logs the administered amount and time-stamps the event for local storage and later synchronization.

ALERT BEHAVIOR. Safety-critical alerts, including low-dose holds or device malfunction, can be configured to bypass the SILENCE detent of the alarm switch (370). Non-critical alerts, such as missed-dose reminders or low-battery warnings, may be silenced.

DATA SYNCHRONIZATION. When connectivity is available, dosing history, guided-intake responses, and sensor data synchronize via Wi-Fi (330) or the cradle interface (320). A paired smartphone via Bluetooth (340) may serve as an alternative gateway. In offline scenarios, the device caches records and performs deferred synchronization when connection resumes.

MATERIALS, MANUFACTURING, AND VARIANTS. The control module housing may be formed from injection-molded polymers such as PC+ABS blends, optionally with internal RF shielding. The barrel (100) may be medical-grade polymer or lightweight metal. The safety seal (160) may be molded from elastomers compatible with the medication type, including butyl rubber, silicone, or EPDM, with optional surface treatments to reduce stiction. The display assembly may be OLED, TFT, or low-power reflective technology. Firmware supports signed updates, diagnostic modes, and customizable UI layouts. Variants may include alternate paddle geometries, different alarm switch placements, expanded sensor inputs, or additional therapy-specific modules, all within the scope of the disclosure.

1. A portable, platform-configurable medical dose-assist module comprising: a housing configured to couple to a medication cartridge; a control module containing a display and at least one user-input device selected from the group consisting of a four-way toggle, a rocker switch, and a microphone/button, and combinations thereof; and a data/power port; a plunger assembly mechanically and electronically linked to the module for dose logging; a separation safety seal located between the plunger assembly and the cartridge interface to reduce cross-contamination; one or more processors operatively coupled to a dosing engine and configured to execute stored instructions to generate dosing guidance; and a dosing engine configured to receive physiologic, behavioral, and contextual inputs comprising at least one of sensor data, manual entry, wellness status, meal intent, activity, concurrent medications, or wearable/phone health metrics, wherein the dosing engine fuses the inputs and applies therapy-specific guardrails or input constraints, and wherein the module is configurable for multiple therapeutic protocols selectable or automatically loaded based on condition-specific parameters; and wherein the module lacks any actuator configured to impart motive force to a pump motor, solenoid, or a plunger for medication delivery; dosing actions are user-initiated.

2. The module of claim 1, wherein the processors are configured to load and execute condition-specific dosing protocols from a stored library.

3. The module of claim 1, wherein guided-intake workflows adapt user prompts to a selected therapeutic category, including drug-specific variables and associated lifestyle factors.

4. The module of claim 1, wherein the control module is configured to generate clinician reports summarizing guided intake and dosing history across multiple therapeutic categories, the reports being exportable in CSV or FHIR-compliant formats.

5. The module of claim 1, wherein the dosing engine applies therapy-specific guardrails comprising minimum-dose holds, maximum per-dose limits, time-window limits, and split-dose scheduling.

6. The module of claim 5, wherein intake composition including macronutrient content is incorporated into guidance such that a portion of a recommended dose may be deferred or a follow-up check scheduled.

7. The module of claim 1, wherein the dosing engine incorporates time-of-day adjustments to dosing parameters across multiple therapeutic protocols.

8. The module of claim 1, wherein the dosing engine incorporates adjustments based on comorbidities, concurrent medications, or therapy-specific contraindications.

9. The module of claim 1, wherein wearable- or phone-sourced wellness metrics including at least one of steps, heart rate, ECG/EKG, or blood pressure are incorporated as optional context to inform temporary target ranges or alert thresholds without overriding stored dosing instructions.

10. The module of claim 1, wherein the control module is configured to log dietary intake and activity patterns via user entry or passive input for incorporation into generated dosing guidance.

11. The module of claim 1, wherein the control module is configured to perform voice-driven nutrient lookup that parses food names and portion sizes and presents portion prompts using household measures, fractions, or weights.

12. The module of claim 11, wherein the nutrient lookup queries a local cache to operate offline and selectively expands coverage via a cloud service when connectivity is available.

13. The module of claim 1, wherein the control module is configured to receive barcode input or menu input from a paired phone as a fallback to voice interaction.

14. The module of claim 1, wherein the guided-intake workflow prompts the user to confirm concurrent medication usage and generates therapy-specific reminders for said medications based on stored dosing schedules.

15. The module of claim 1, wherein the control module is configured to synchronize stored data directly over Wi-Fi without requiring a smartphone intermediary.

16. The module of claim 1, wherein the module operates independently of a smartphone by caching data on-device, storing interaction and usage data locally, and performing deferred synchronization via Wi-Fi or a cradle connection.

17. The module of claim 1, wherein a separation alarm is configured to trigger when the module loses connection to a paired cradle or connected sensor interface, the alarm generating a local and/or wireless alert.

18. The module of claim 1, further comprising a security module configured to: enable PIN-based access with retry back-off; encrypt stored data at rest; verify signed firmware and block rollback to unsigned versions; and support over-the-air firmware updates via a cradle connection or a paired smartphone gateway.

19. The module of claim 18, further comprising a remote-wipe control configured to perform either a device-only wipe or a device-plus-cradle wipe, with default retention of last-synced data on the cradle unless a complete wipe is selected.

20. The module of claim 18, wherein the security module enables PIN reset through a user-authenticated email process routed via a paired smartphone.

21. The module of claim 1, further comprising a location-assist function configured to identify module proximity and to trigger a visual, audio, or wireless alert when the module is out of range of a paired cradle or sensor interface.

22. The module of claim 1, wherein a plunger paddle of the plunger assembly is shaped for one-hand actuation and includes ergonomic features configured to accommodate user tremor.

23. The module of claim 1, wherein the display window is configurable to render either (a) a single high-contrast line for large-font readability or (b) two lines for expanded information display, wherein one line presents the suggested dose or action plan and the other line presents a rationale or supplemental data.

24. The module of claim 1, further comprising a cradle interface having a simplified keypad configured to enable manual entry of time adjustments, biometric or therapy-related values, Wi-Fi credentials, and healthcare-provider link codes, the cradle being further configured to assume limited input/output functionality including error prompts and data recovery when the module is nonresponsive; and wherein the housing is not a replacement pen cap.

25. The module of claim 1, wherein the user interface is multilingual, with guided-intake prompts and on-screen output available in multiple languages selectable by the user or by a healthcare provider.

26. The module of claim 1, further comprising a parental or supervisory monitoring mode configured to log, transmit, and confirm dosing actions for pediatric or dependent patients.

27. The module of claim 1, wherein the dosing engine is configured to generate therapy recommendations for user review and approval without initiating closed-loop therapy delivery.

28. The module of claim 1, wherein the cartridge interface is configured to accept a 3 mL, 100-unit O-ring-type cartridge suitable for subcutaneous delivery.

29. A method for providing multi-condition medication dose assistance, comprising: receiving, at a portable dose-assist module, physiologic data from one or more sensors or external sources; accepting user-supplied health-status and dosage-related inputs via keypad, touchscreen, or voice input; applying confidence weighting or preferring a direct-measurement value when it materially differs from sensor-derived data; integrating the inputs with stored dosing instructions, duration-of-action parameters, wellness-status prompts, activity information, concurrent medications, and condition-specific variables for a plurality of therapeutic areas to generate a composite guidance output; and outputting a therapy recommendation for user review and approval without initiating closed-loop therapy delivery.

30. A non-transitory computer-readable medium storing instructions that, when executed by one or more processors, cause execution of the method of claim 29.
